(19) United States
(12) Patent Application Publication
(10) Pub. No.: US 2020/0303043 A1
(43) Pub. Date: Sep. 24, 2020

(54) SYSTEM, METHOD AND SOFTWARE FOR CALCULATION OF CANNABIS DRUG EFFICIENCY INDEX (CDEI)

(71) Applicant: Pathway Rx, Inc., Lethbridge (CA)

(72) Inventors: Boseon Byeon, Lethbridge (CA); Yaroslav Ilnytskyy, Lethbridge (CA); Olga Kovalchuk, Lethbridge (CA); Nikolay Mikhaylovich Borisov, Moscow (RU); Igor Kovalchuk, Lethbridge (CA)

(21) Appl. No.: 16/819,190

(22) Filed: Mar. 16, 2020

Related U.S. Application Data
(60) Provisional application No. 62/820,897, filed on Mar. 20, 2019.

(51) Int. Cl. G16C 60/00; A61K 45/06; A61K 36/185; G16H 70/40; G16H 20/10

(52) U.S. Cl. CPC G16C 60/00; A61K 45/06; G16H 20/10; G16H 70/40; A61K 36/185

(57) ABSTRACT

The present invention provides systems, methods and software for assessment of the personalized efficacy of cannabis drug for treatment of various diseases, disorders, syndromes and conditions, based on analysis of high-throughput gene expression profiling. According to signaling pathway topology, the gene expression profiles are convoluted into signaling pathway activities using an SPIA method. The drug action as evaluated by comparison between samples of the disease states before and after treatment with cannabis drug, as well as control samples and providing an individual with a treatment having a cannabis drug efficiency index (CDEI) greater than 0.

### FIELD OF THE INVENTION

The present invention relates generally to systems and methods of analysis of biological pathway data, and more specifically to systems and methods for predicting personalized cannabis drug efficiency indices.

### BACKGROUND OF THE INVENTION

In the twentieth century, enormous strides were made in combatting infectious diseases, in their detection and drugs to treat them. The major problem in the medical world has thus shifted from treating acute diseases to treating chronic diseases. Over the last few decades, with the advent of genetic engineering, much research and funding has been invested in genomics and gene-based personalized medicine. A need has arisen to develop diagnostic tools for use in the characterization of personalized aspects of chronic diseases.

Intracellular signaling pathways (SPs) regulate numerous processes involved in normal and pathological conditions including development, growth, aging and cancer. Many bioinformatics tools have been developed, which analyze SPs. Many intracellular signaling pathways or maps are available at online websites. Additionally, they can be found in publications, such as, but not limited to Cooper et al, 2000 and Krauss, 2008.

The information relating to signaling pathway activation (SPA) can be obtained from the massive proteomic or transcriptomic data. Although the proteomic level may be somewhat closer to the biological function of SPA, the transcriptomic level of studies today is far more feasible in terms of performing experimental tests and analyzing the data.

Intracellular signaling pathways (SPs) regulate numerous processes involved in normal and pathological conditions including development, growth, aging and cancer. Many bioinformatics tools have been developed recently that analyze SPs. However, none of them makes it possible to efficiently do the high-throughput quantification of pathway activation scores for the individual biological samples. Here we propose a method for quick, informative and large-scale screening of changes in signaling pathway activation (SPA) in cells and tissues. These changes may reflect various differential conditions like differences in physiological state, aging, disease, treatment with drugs, infections, media composition, additives, etc. One of the potential applications of SPA studies may be in utilizing mathematical algorithms to identify and rank the medicines based on their predicted efficacy.

The information about SPA can be obtained from the massive proteomic or transcriptomic data. Although the proteomic level may be somewhat closer to the biological function of SPA, the transcriptomic level of studies today is far more feasible in terms of performing experimental tests and analizing the data. The transcriptomic methods like Next-generation sequencing (NGS) or microarray analysis of RNA can routinely determine expression levels for all or virtually all human genes (Shirane, 2004). Transcriptome profiling may be performed for the minute amount of the tissue sample, not necessarily fresh, but also for the clinical formalin-fixed, paraffin-embedded (FFPE) tissue blocks. For the molecular analysis of cancer, gene expression can be interpreted in terms of abnormal SPA features of various pro- and antimitotic signaling pathways. Such analysis may improve further decision-making process of treatment strategy selection by the clinician.

Pro- and antimitotic SPs that determine various stages of cell cycle progression remained in the spotlight of the computational biologists for more than a decade (Kholodenko, 1999; Borisov, 2009; Kuzmina, 2011). Today, hundreds of SPs and related gene product interaction maps that show sophisticated relationships between the individual molecules, are catalogued in various databases like UniProt (The UniProt consortium, 2011), HPRD (Mathivanan, 2006), QIAGEN SABiosciences (SABiosciences), WikiPathways (Bauer-Mehren, 2009), Ariadne Pathway Studio (Nikitin, 2004), SPIKE (Elkon, 2008), Reactome (Haw, 2012), KEGG (Nakaya, 2013), etc.

One group of bioinformatic approaches integrated the analysis of transcriptome-wide data with the models employing the mass action law and Michaelis-Meten kinetics (Yizhak, 2013). These methods which were developing during last fifteen years, however, remained purely fundamental until recently, primarily, because of the multiplicity of interaction domains in the signal transducer proteins that enormously increase the interactome complexity (Borisov, 2008; Conzelman, 2006). Secondly, a considerable number of unknown free parameters, such as kinetics constants and/or concentrations of protein molecules, significantly complicated the SPA analysis. Yizhak et al. (2013) suggested that the clinical efficiency of several drugs, e.g. geroprotectors, may be evaluated as the ability to induce the kinetic models of the pathways into the steady state. However, protein-protein interactions were quantitatively characterized in detail only for a tiny fraction of SPs. This approach is also time-consuming since to process each transcriptomic dataset it requires extensive calculations for the kinetic models (Yizhak, 2013).

However, all the contemporary bioinformatical methods that were proposed for digesting large-scale gene expression data followed by recognition and analysis of SPs, have an important disadvantage. They do not allow tracing the overall pathway activation signatures and quantitively estimate the extent of SPA (Hwang, 2012; Kuzmina, 2011; Yizhak, 2013). This may be due to lack of the definition of the specific roles of the individual gene products in the overall signal transduction process, incorporated in the calculation matrix used to estimate SPA.

US2008254497A provides a method of determining whether tumor cells or tissue is responsive to treatment with an ErbB pathway-specific drug. In accordance with the invention, measurements are made on such cells or tissues to determine values for total ErbB receptors of one or more types, ErbB receptor dimers of one or more types and their phosphorylation states, and/or one or more ErbB signaling pathway effector proteins and their phosphorylation states. These quantities, or a response index based on them, are positively or negatively correlated with cell or tissue responsiveness to treatment with an ErbB pathway-specific drug. In one aspect, such correlations are determined from a model of the mechanism of action of a ErbB pathway-specific drug on an ErbB pathway. Preferably, methods of the invention are implemented by using sets of binding compounds having releasable molecular tags that are specific for multiple components of one or more complexes formed in ErbB pathway activation. After binding, molecular tags are released and separated from the assay mixture for analysis.

U.S. Pat. No. 8,623,592 discloses methods for treating patients which methods comprise methods for predicting responses of cells, such as tumor cells, to treatment with therapeutic agents. These methods involve measuring, in a sample of the cells, levels of one or more components of a cellular network and then computing a Network Activation State (NAS) or a Network Inhibition State (NIS) for the cells using a computational model of the cellular network. The response of the cells to treatment is then predicted based on the NAS or NIS value that has been computed. The invention also comprises predictive methods for cellular responsiveness in which computation of a NAS or NIS value for the cells (e.g., tumor cells) is combined with use of a statistical classification algorithm. Biomarkers for predicting responsiveness to treatment with a therapeutic agent that targets a component within the ErbB signaling pathway are also provided.

U.S. Pat. No. 9,095,554 B2 discloses compositions and methods for the breeding, production, processing and use of specialty cannabis.

There thus remains an unmet urgent and increasing need to provide effective personalized non-toxic disease therapies, and models for selecting a personalized optimal therapy for an individual.

### SUMMARY OF THE INVENTION

The present invention provides systems, methods and software for assessment of the personalized efficacy of cannabis drug (oil, gel caps, topical etc.) for treatment of various diseases, disorders, syndromes and conditions, based on analysis of high-throughput gene expression profiling. According to signaling pathway topology, the gene expression profiles are convoluted into signaling pathway activities using an SPIA method. The drug action as evaluated by comparison between samples of the disease states before and after treatment with cannabis drug, as well as control samples and providing an individual with a treatment having a cannabis drug efficiency index (CDEI) greater than 0.

It is an object of some aspects of the present invention to provide systems and methods for comparison of high-throughput gene expression profiles and signaling pathway activation profiles between case and normal reference states for prediction of cannabis drug action on individual patients.

In other embodiments of the present invention, a method and system is described for providing personalized analyses of optimized drug efficacy prediction per individual patients' profiles, such as genetic profiles.

In additional embodiments for the present invention, a method and system for predicting optimized drug profiles for treating a specific patient disease or disorder are provided.

In further embodiments of the present invention, a method and system for predicting optimized drug profiles for treating a specific patient proliferative disease or disorder are provided.

There is thus provided according to an embodiment of the present invention, a method for ranking efficiency of Cannabis drugs, the method including;
a. calculating a Signaling Pathway Impact analysis (SPIA) for each drug for each biological pathway;
b. determining a mean weighted SPIA_{μ}, wherein SPIA_{μ}=mean(SPIA)·wₚ;
c. calculating a cannabis drug efficiency index (CDEI) for each drug for a specific disease, wherein CDEI=2 ((|t_{U}|/(|t_{T}|+|t_{U}|)−0.5); and
d. ranking the drugs according to highest CDEI for a group of individual patients.

Additionally, according to another embodiment of the present invention, the method further includes treating an individual patient suffering from the specific disease with at least one drugs of one or more high CDEIs, wherein the high CDEI>0.

Furthermore, according to another embodiment of the present invention, the at least one drug alleviates, cures or attenuates the specific disease.

Further, according to another embodiment of the present invention, the high CDEI>0 is a highest ranking CDEI.

Yet further, according to another embodiment of the present invention, the disease is proliferative disease or disorder.

Additionally, according to another embodiment of the present invention, the proliferative disease or disorder is cancer.

Further, according to another embodiment of the present invention, the CDEI>=0.2.

Importantly, according to another embodiment of the present invention, A method according to claim 1, wherein the CDEI>=0.5 and <1.

Additionally, according to another embodiment of the present invention, the wₚ=([number of case samples with positive SPIA score]/[total number of case samples).

Further, according to another embodiment of the present invention, the wₚ=([number of case samples with negative SPIA score]/[total number of case samples).

There is thus provided according to another embodiment of the present invention, a computer software product, the product configured for ranking efficiency of Cannabis drugs, the product including a computer-readable medium in which program instructions are stored, which instructions, when read by a computer, cause the computer to;
a. calculate a Signaling Pathway Impact analysis (SPIA) for each drug for each biological pathway;
b. determine a mean weighted SPIA_{μ} wherein SPIA_{μ}=mean(SPIA)·wₚ;
c. calculate a cannabis drug efficiency index (CDEI) for each drug for a specific disease, wherein CDEI=2 ((|t_{U}|/(|t_{T}|+|t_{U}|)−0.5); and
d. rank the drugs according to highest CDEI for a group of individual patients.

There is thus provided according to another embodiment of the present invention, a system for ranking efficiency of Cannabis drugs, the system including;
a. a processor adapted to activate a computer-readable medium in which program instructions are stored, which instructions, when read by a computer, cause the processor to;
   i. calculate a Signaling Pathway Impact analysis (SPIA) for each drug for each biological pathway;
   ii. determine a mean weighted SPIA_{μ} wherein SPIA_{μ}=mean(SPIA)·wₚ;
   iii. calculate a cannabis drug efficiency index (CDEI) for each drug for a specific disease, wherein CDEI=2 ((|t_{U}|/(|t_{T}|+|t_{U}|)−0.5); and
   iv. rank the drugs according to highest CDEI for a group of individual patients;
b. a memory for storing the Signaling Pathway Impact analysis (SPIA) for each a plurality of biological pathways; and
c. a display for displaying data associated with the CDEI.

There is thus provided according to another embodiment of the present invention, a bioinformatics method for ranking Cannabis drugs, the method including;
a. calculating a Signaling Pathway Impact analysis (SPIA) for each drug for each biological pathway;
b. determining a mean weighted SPIA_{μ} wherein SPIA_{μ}=mean(SPIA)·wₚ;
c. calculating a cannabis drug efficiency index (CDEI) for each drug for a specific disease, wherein CDEI=2 ((|t_{U}|/(|t_{T}|+|t_{U}|)−0.5); and
d. ranking the drugs according to highest CDEI for a group of individual patients.

Additionally, according to another embodiment of the present invention, the method is performed on a plurality of ethnic groups to determine an optimized ranking of the disease-protective drugs for each ethnic group.

Furthermore, according to another embodiment of the present invention, the method is performed for an individual to determine an optimized ranking of the disease-protective drugs for the individual.

Further, according to another embodiment of the present invention, the disease is cancer.

Moreover, according to another embodiment of the present invention, n the biological pathways are signaling pathways.

Additionally, according to another embodiment of the present invention, a data is obtained from studies on samples of the individual patients.

Furthermore, according to another embodiment of the present invention, the samples are bodily samples selected from the group consisting of a blood sample, a urine sample, a biopsy, a hair sample, a nail sample, a breathe sample, a saliva sample and a skin sample.

There is thus provided according to another embodiment of the present invention, a method for ranking efficiency of Cannabis drugs, the method including;
a. calculating a Signaling Pathway Impact analysis (SPIA) for each drug for each biological pathway;
b. determining a mean weighted SPIA wherein SPIA_{μ}=mean(SPIA)·wₚ;
c. calculating a cannabis drug efficiency index (CDEI) for each drug for a specific disease, wherein CDEI=2 ((|t_{U}|/(|t_{T}|+|t_{U}|)−0.5); and
d. ranking the drugs according to highest CDEI for an individual patient suffering for a disease.

It is an object of some aspects of the present invention, to provide a proliferative transcriptome in which the transcribed genes in subjects with a proliferative disease or disorder relative (sick subjects) to healthy individuals are compared to define a set first of genes which are more strongly expressed (activated) in sick people or subjects relative to healthy people or subjects and a second set of genes (repressed) which are less strongly expressed in sick people or subjects relative to healthy people or subjects.

It is an object of some aspects of the present invention to provide effective non-toxic cancer therapies, methods for their manufacture and methods of cancer treatment.

In some embodiments of the present invention, improved methods and apparatus are provided for effective non-toxic cancer therapies, methods for their manufacture and methods of cancer treatment.

It is an object of some aspects of the present invention to provide compositions for improving wellness in a human or mammalian organism.

It is another object of some aspects of the present invention to provide compositions for preventing or treating diseases or disorders in a human or mammalian organism.

The compositions and dosage forms of the present invention are useful in promoting health and preventing or treating a large number of disorders in human patients and other mammalian subjects.

In additional embodiments of the present invention, compositions and methods are provided for treating and/or preventing proliferative disorders.

In additional embodiments of the present invention, compositions and methods are provided for treating and/or preventing cancer.

In additional embodiments of the present invention, compositions and methods are provided for treating and/or preventing skin disorders.

The present invention is directed to compositions and methods for treating disorders, in general, and more particularly, proliferative disorders and diseases. The compositions of the present invention may be used for improving wellness of a human or mammalian subject. Additionally, the compositions of the present invention may be used to treat any disorder or ailment in a human patient or mammalian subject. Furthermore, the compositions of the present invention may conveniently be used in conjunction with a drug to treat any disorder or ailment in a human patient or mammalian subject.

In other some embodiments of the present invention, a method is described for providing effective non-toxic cancer therapies, derived from Cannabis sativa (marijuana and/or hemp) plants, from now on referred to as “cannabis”.

The present invention further provides new unique cannabis lines, extracts, dried powders from the extracts, compositions comprising the powders or parts thereof, compounds derived therefrom, pharmaceutical compositions comprising the compound(s) and methods for their use in anti-cancer therapies and modalities. The method includes generation of unique lines, whole plant extract preparation, treating cancer cells and normal cells with extracts in amount sufficient to kill cancer cells while sparing normal (non-proliferative) ones. The modulation of cell proliferation, growth and death results in efficient elimination of cancer cells in response to the anti-cancer therapies and modalities of the present invention.

Cannabis has been suggested to harbor anti-cancer potential, however it has only gained much attention in the last two decades. While the most prevalent cannabinoids—Δ9-THC, cannabidiol (CBD), and cannabinol (CBN) were shown to affect cellular growth and proliferation, it is well-known that whole plant extracts have a milieu of other active molecules (terpenes, etc). Some terpenes may also have cytotoxic properties, thus full extracts exhibit the so-called combined “entourage effects”.

The present invention provides new Cannabis sativa (marijuana and hemp) and combinations thereof and extracts thereof and method of using them as a means to kill cancer cells while sparing normal ones. The disclosure also provides methods of modulating cell cycle, cell growth and cell death through the application of extracts of novel cannabis lines on cancer cell line models.

The present invention further provides new Cannabis sativa lines and extracts and method of using them as a means to kill cancer cells while sparing normal ones. The disclosure also provides methods of modulating cell cycle, cell growth and cell death through the application of extracts of novel cannabis lines on cancer cell line models.

Accordingly, the disclosure provides a means for modulating gene expression by cannabis and/or hemp extracts (e.g., in skin tissues after exposure to UV light, a known carcinogen) by providing amounts sufficient to modulate gene expression where modulation of gene expression results in suppression of cell growth.

Further disclosed are over a hundred freshly prepared extracts of Cannabis sativa lines and a plurality of identified extracts displaying very pronounced anti-cancer activity.

The present invention provides systems, methods and software for running a bioinformatics tool for predicting the clinical efficiency of cannabis drug application to individual persons.

As an input data, the CDEI operates with the results of various “omics” data stemming from the cells of individual patients, as well as from the healthy individuals. These data may include transcriptomic (e.g. performed with either next-generation sequencing or microarray mRNA hybridization), non-coding-RNAomic, proteomic, epigenomic etc.

The data of full mRNA/protein abundance are integrated by CDEI into the assessment values for activation of different cellular pathways (signalome).

The topology for signaling, metabolic, cytoskeleton etc. pathways was borrowed from the database of QIAGEN SABiosciences (URL: https://www.qiagen.com/ru/shop/genes-and-pathways/pathway-central/).

These high-throughput gene expression data are converted according to a CDEI approach, to more aggregated values of molecular pathway activities using the signaling pathway impact analysis, Signaling Pathway Impact analysis—SPIA method (Tarca et al., 2009). Among other methods for gene-expression based assessment of signaling pathway activation, including TAPPA (Gao and Wang, 2007), topology-based score (TB) (Ibrahim et al., 2012), Pathway-Express (PE) (Draghici et al., 2007), and OncoFinder (Buzdin et al., 2014), the SPIA (Tarca et al., 2009) approach showed the best statistical performance during the comparison of pathway-based to gene-based values (FIG. 3; borrowed from (Borisov et al., 2017)).

Additionally, according to an embodiment of the present invention, the method is quantitative.

Further, according to an embodiment of the present invention, the method is qualitative.

Yet further, according to an embodiment of the present invention, the subject is a vertebrate.

Moreover, according to an embodiment of the present invention, the subject is mammalian.

Additionally, according to an embodiment of the present invention, the subject is human.

Furthermore, according to an embodiment of the present invention, the sick subject suffers from an acute or chronic disease or disorder, such as an inflammatory condition like rheumatoid arthritis or osteoarthritis, irritable bowel disease, psoriasis, eczema or any other disease, such as autoimmune diseases, proliferative disease or disorder.

In some cases, according to an embodiment of the present invention, the proliferative disease or disorder is cancer.

Additionally, according to an embodiment of the present invention, the method is performed on a plurality of ethnic groups to determine an optimized ranking of the disease-protective drugs for each ethnic group.

Furthermore, according to an embodiment of the present invention, the method is performed for an individual to determine an optimized ranking of the disease-protective drugs for the individual.

Further, according to an embodiment of the present invention, the mapping step further includes mapping each of the plurality of biological pathways for the activation strength and the down-regulation strength.

Furthermore, according to an embodiment of the present invention, the biological pathways are signaling pathways.

Additionally, according to an embodiment of the present invention data is obtained from studies on the samples of the subjects.

Further, according to an embodiment of the present invention, the samples are bodily samples selected from the group consisting of a blood sample, a urine sample, a biopsy, a hair sample, a nail sample, a breathe sample, a saliva sample and a skin sample.

Importantly, according to an embodiment of the present invention, wherein the method is effective in helping to slow down various diseases.

Additionally importantly, according to an embodiment of the present invention, the method is effective in helping to cure various conditions.

Additionally, according to an embodiment of the present invention, a bioinformatics tool is provided for predicting the clinical efficiency of cannabis drug application to individual persons.

The present invention will be more fully understood from the following detailed description of the preferred embodiments thereof, taken together with the drawings and appendices.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic illustration of a system for running a bioinformatics tool for predicting the clinical efficiency of cannabis drug application to an individual, in accordance with an embodiment of the present invention;

FIG. 2 is a simplified schematic illustration of databases in the system of FIG. 1, in accordance with an embodiment of the present invention;

FIG. 3 provides a graphical comparison of a data aggregation effect, R, for five pathway activation scoring methods (OncoFinder (OF)), TAPPA, TBScore (TB), Pathway-Express (PE), and SPIA on a renal carcinoma dataset (Borisov et al., 2017);

FIG. 4 is a simplified flowchart of a method for running a bioinformatics tool for predicting the clinical efficiency of cannabis drug application to an individual, in accordance with an embodiment of the present invention; and

FIG. 5 is a simplified bar chart of oral and intestinal CDEI scores for nine different cannabis cultivars, in accordance with an embodiment of the present invention.

In all the figures similar reference numerals identify similar parts.

### DETAILED DESCRIPTION OF THE EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

### Overview of Signaling Pathway Impact Analysis (SPIA) Method

Imagine a pathway graph, G(V, E), where V={g₁, g₂, K, gₙ} is the set of graph nodes (vertices), and E={(gᵢ, gⱼ)|genes gᵢ and gⱼ interact} is the set of graph edges. The adjacency matrix is defined as follows, aᵢⱼ=1, if i=j or (gᵢ, gⱼ)∈E, and aᵢⱼ=0, if (gᵢ, gⱼ)∉E.

Consider then values called perturbation factors (PF) for the all genes g of the pathway K,

$$P F ( g ) = Δ E ( g ) + {\sum }_{γ ∈ {U}_{g}} {β}_{γ g} \frac{P F ( γ )}{{n}_{down} ( γ )} .$$

Here ΔE(_{g}) is the signed log-fold-change of gene g expression level in a given sample compared with the average value for the pool of normal samples. The latter term expresses the summation over all the genes γ that belong to the set U_{g} of the upstream genes for the gene g. The value of n_{down}(γ) denotes the number of downstream genes for gene γ. The weight factor β_{γg} indicates the interaction type between γ and g: β_{γg}=1 if γ activates g, and β_{γg}=−1 when γ inhibits g. The search for upstream/downstream genes is performed according to the depth-first search method (Even and Guy Even, 2012).

To obtain an estimator for pathway perturbation that is positive for an up-regulated and negative for a down-regulated pathway, use the second term in formula for the perturbation factor (PF) from the previous paragraph, resulting in the accuracy value, Acc(g)=PF(g)−ΔE(g). It can be shown that this accuracy vector may be expressed as follows,

$$Acc = B \cdot {( I - B )}^{- 1} \cdot Δ E , where$$

$$B = ( \begin{matrix}\frac{{β}_{11}}{{n}_{down} ( {g}_{1} )} & \frac{{β}_{12}}{{n}_{down} ( {g}_{2} )} & Λ & \frac{{β}_{1 n}}{{n}_{down} ( {g}_{n} )} \\ \frac{{β}_{21}}{{n}_{down} ( {g}_{1} )} & \frac{{β}_{22}}{{n}_{down} ( {g}_{2} )} & Λ & \frac{{β}_{2 n}}{{n}_{down} ( {g}_{n} )} \\ \frac{{β}_{n 1}}{{n}_{down} ( {g}_{1} )} & \frac{{β}_{n 2}}{{n}_{down} ( {g}_{2} )} & Λ & \frac{{β}_{nn}}{{n}_{down} ( {g}_{n} )}\end{matrix} ) ,$$

I is the identity matrix, and

$$Δ E = ( \begin{matrix}Δ E ( {g}_{1} ) \\ Δ E ( {g}_{2} ) \\ Λ \\ Δ E ( {g}_{n} )\end{matrix} ) .$$

The overall score for pathway pertubation calculated as: SPIA=Σ_{g}Acc(g) (Tarca et al., 2009).

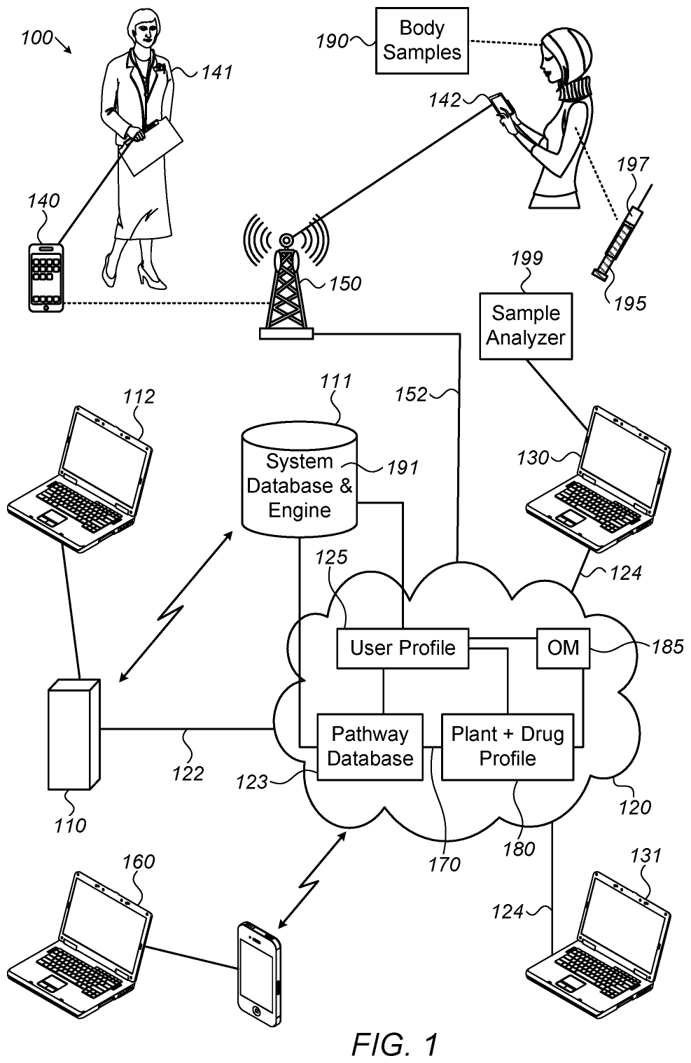

Reference is now made to FIG. 1, which is a simplified schematic illustration of a system 100 for running a bioinformatics tool for predicting the clinical efficiency of cannabis drug application to an individual, in accordance with an embodiment of the present invention.

System 100 typically includes a server utility 110, which may include one or a plurality of servers and one or more control computer terminals 112 for programming, trouble-shooting servicing and other functions. Server utility 110 includes a system engine 111 and database, 191. Database 191 comprises a user profile database 125, a at least one online database 123, stored in cloud 120, for example (or on a server) and a drug profile database 180.

Depending on the capabilities of a mobile device, system 100 may also be incorporated on a mobile device that synchronizes data with a cloud-based platform.

The drug profile database comprises data relating to a large number of drugs for controlling and treating cancer. For each type of drug, the dosage values, pharmo-kinetic data and profile, pharmodynamic data and profiles are included.

The drug profile database further comprises data of drug combinations, including dosage values pharmo-kinetic data and profile, pharmodynamic data and profiles.

A medical professional, research personnel or patient assistant/helper/career 141 is connected via his/her mobile device 140 to server utility 110. The patient, subject or child 143 is also connected via his/her mobile device 142 to server utility 110. In some cases, the subject may be a mammalian subject, such as a mouse, rat, hamster, monkey, cat or dog, used in research and development. In other cases, the subject may be a vertebrate subject, such as a frog, fish or lizard. The patient or child is monitored using a sample analyzer 199. Sample analyzer 199, may be associated with one or more computers 130 and with server utility 110. Computer 130 and/or sample analyzer 199 may have software therein for performing the “CDEI method” of the present invention. The outputs of the software may be displayed, such as a CDEI ranking table 132, described in further detail hereinbelow and in the appendices.

Typically, online database 123 (FIG. 1), generated by the software of the present invention, is stored locally and/or in cloud 120 and/or on server 110.

The sample analyzer may be constructed and configured to receive a solid sample 190, such as a biopsy, a hair sample or other solid sample from patient 143, and/or a liquid sample 195, such as, but not limited to, urine, blood or saliva sample. The sample may be extracted by any suitable means, such as by a syringe 197.

The patient, subject or child 143 may be provided with a drug (not shown) by health professional/research/doctor 141.

System 100 further comprises an outputting module 185 for outputting data from the database via tweets, emails, voicemails and computer-generated spoken messages to the user, careers or doctors, via the Internet 120 (constituting a computer network), SMS, Instant Messaging, fax through link 122.

Users, patients, health care professionals or customers 141, 143 may communicate with server 110 through a plurality of user computers 130, 131, or user devices 140, 142, which may be mainframe computers with terminals that permit individual to access a network, personal computers, portable computers, small hand-held computers and other, that are linked to the Internet 120 through a plurality of links 124. The Internet link of each of computers 130, 131, may be direct through a landline or a wireless line, or may be indirect, for example through an intranet that is linked through an appropriate server to the Internet. System 100 may also operate through communication protocols between computers over the Internet which technique is known to a person versed in the art and will not be elaborated herein.

Users may also communicate with the system through portable communication devices such as mobile phones 140, communicating with the Internet through a corresponding communication system (e.g. cellular system) 150 connectable to the Internet through link 152. As will readily be appreciated, this is a very simplified description, although the details should be clear to the artisan. Also, it should be noted that the invention is not limited to the user-associated communication devices—computers and portable and mobile communication devices—and a variety of others such as an interactive television system may also be used.

The system 100 also typically includes at least one call and/or user support and/or tele-health center 160. The service center typically provides both on-line and off-line services to users. The server system 110 is configured according to the invention to carry out the methods of the present invention described herein.

It should be understood that many variations to system 100 are envisaged, and this embodiment should not be construed as limiting. For example, a facsimile system or a phone device (wired telephone or mobile phone) may be designed to be connectable to a computer network (e.g. the Internet). Interactive televisions may be used for inputting and receiving data from the Internet. Future devices for communications via new communication networks are also deemed to be part of system 100. Memories may be on a physical server and/or in a virtual cloud.

A mobile computing device may also embody a non-synced or offline copy of memories, copies of pathway cloud data, user profiles database, drug profiles database and execute the system, engine locally.

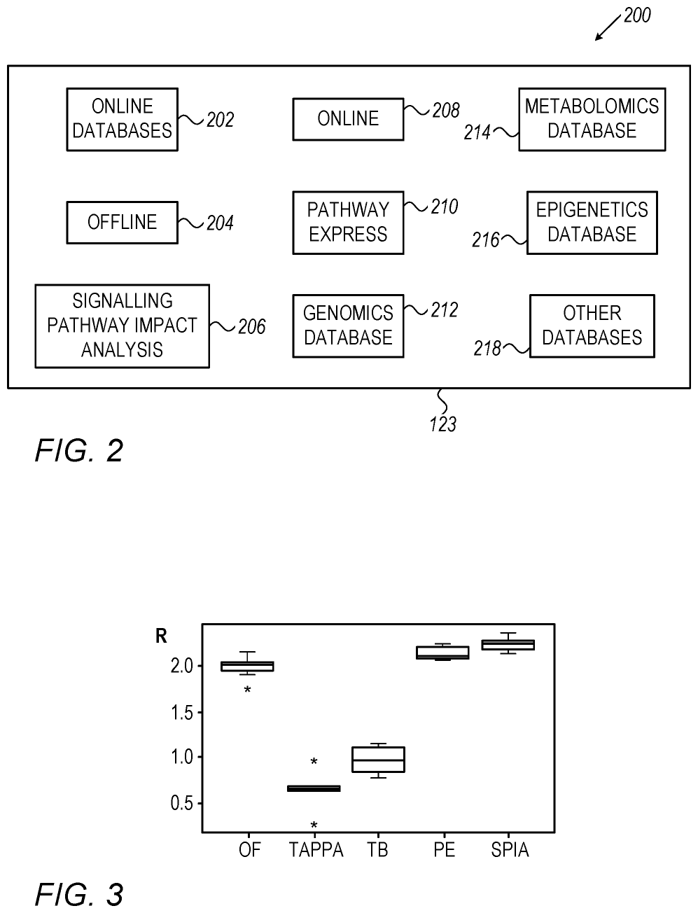

FIG. 2 is a simplified schematic illustration of databases 123 in the system of FIG. 1, in accordance with an embodiment of the present invention. The databases may comprise one or more online databases 202, an offline database 204, a signaling pathway impact (SPIA) database 206, an online database 208 a pathway express database 210, a genomics database 212, a metabolomics database 214, an epigenetics database 216 and any other appropriate, publically or privately accessible databases 218.

FIG. 3 provides a graphical comparison of a data aggregation effect, R, for five pathway activation scoring methods (OncoFinder (OF)), TAPPA, TBScore (TB), Pathway-Express (PE), and Signaling Pathway Impact analysis (SPIA) on a renal carcinoma dataset (Borisov et al., 2017).

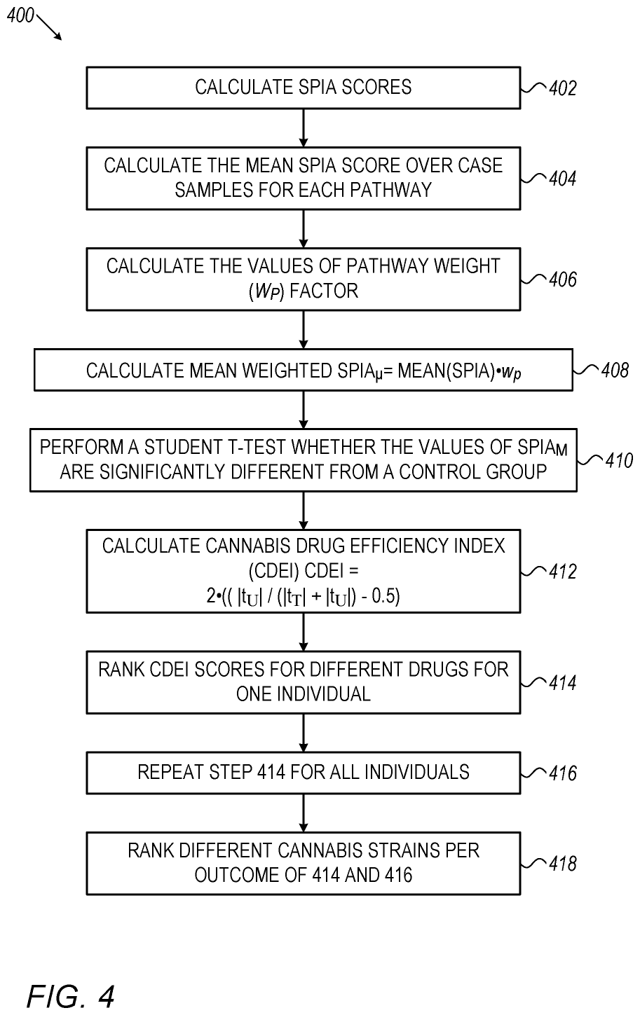

FIG. 4 is a simplified flowchart of a method 400 for running a bioinformatics tool for predicting the clinical efficiency of cannabis drug application to an individual, in accordance with an embodiment of the present invention.

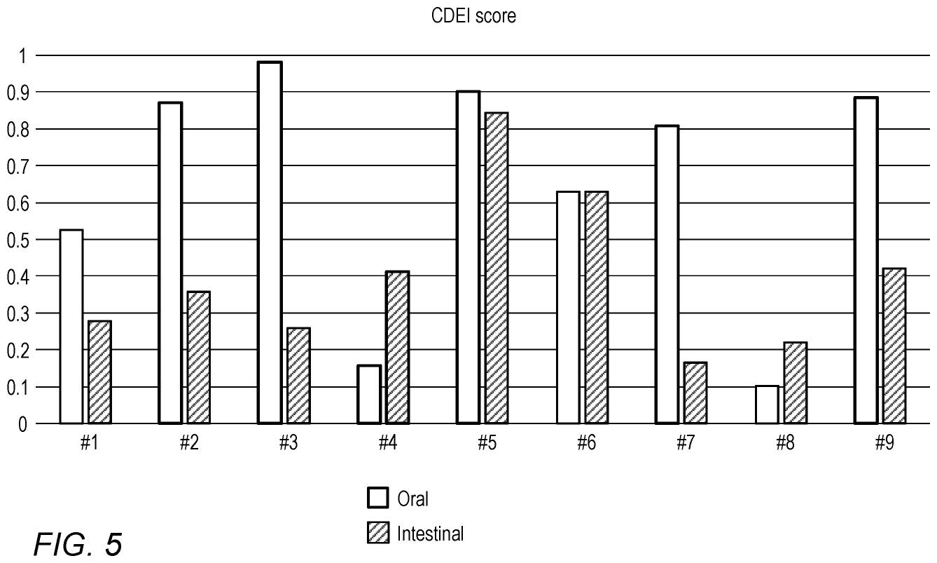

FIG. 5 shows the CDEI scores calculated from treatment of oral and intestinal tissues with extracts prepared from flowers from nine different cannabis cultivars, in accordance with an embodiment of the present invention.

### Calculation of Cannabis Drug Efficiency Index (CDEI)

The following SPIA-based algorithm is proposed for evaluation of the cannabis drug efficiency index.

In a calculating SPIAs scores step 402, at least one SPIA score is calculated for an individual. The overall score for pathway pertubation calculated as: SPIA=Σ_{g}Acc(g) (Tarca et al., 2009). In order to avoid infinite values during log-fold-change calculations, added the value of 1 to all expression values for the datasets, of which any gene has all the control values of zero. In some cases SPIA scores are calculated using Matlab software. Calculating SPIAs scores step 402 may be repeated for an individual for several different pathways. Additionally or alternatively, the SPIA score may be calculated for an individual for a specific pathway a number of times for one sample and/or a number of times for different samples, sampled with predefined time intervals.

In a calculating a mean SPIA score step 404, a mean SPIA score for all case samples for each pathway is calculated. This may include multiple samples from multiple individuals for each individual pathway.

Thereafter a calculating a pathway weight (wp) factor step 406 is performed for each pathway. This is performed as follows:

For pathways with positive mean SPIA score of case samples,

wₚ=([number of case samples with positive SPIA score]/[total number of case samples])

For pathways with negative mean SPIA score of case samples,

wₚ=([number of case samples with negative SPIA score]/[total number of case samples]).

In a calculating a mean weighted SPIA step 408, a mean SPIA score of case samples in each pathway is multiplied by the weight factor from step 406, in accordance with:

SPIA_{μ}=mean(SPIA)·wₚ.

The pathways with the mixed signs have the lower weights than the pathways with the consistent signs. Here, SPIAμ is the adjusted mean score of case samples in each pathway.

In a statistical analysis step 410, one or more statistical tests, such as, but not limited to, a Student t-test is performed to analyze whether the values of SPIAμ are different from 0 (control group). For the control samples, the values of SPIAμ are clearly equal to 0.

In a calculating a cannabis drug efficiency step 412, the following constraints/conditions should be satisfied:

1.—Untreated case (U), i.e. the pathological state before drug application, should be far from the control (C).

2.—Treated case (T), i.e. the pathological state after drug application, should be close to the control.

To comply with these requirements, a cannabis drug efficiency index (CDEI) is defined as follows:
   |t_{U}|=absolute t-statistic for the Student's test for U-vs-C profiles
   |t_{T}|=absolute t-statistic for the Student's test for T-vs-C profiles

CDEI=2((|t_{U}/(|t_{T}|+|t_{U}|)−0.5)

Note that the value of CDEI has the following properties:
CDEI is a value between −1 and 1.
CDEI is 0 if |t_{T}| and |t_{U}| are same, which means no drug efficiency.
CDEI is 1 if |t_{T}| is 0, which means a perfect drug efficiency.
CDEI is a value greater than 0 if |t_{T}| is smaller than |t_{U}|, which means the positive efficiency.
CDEI is a value less than 0 if |t_{T}| is larger than |t_{U}|, which means the negative efficiency.

Thus, the value of CDEI satisfies the conditions (*).

Mean score of case samples in each pathway is first calculated/adjusted and then the set of the mean scores in each data set are t-tested for the difference from 0 (i.e. one sample t-test). So a t-statistic is calculated for each data set and the CDEI is one value for each case sample data set. Refer to the CDEI._{R script} under the cdei directory of software.

### Example of CDEI Calculations

This approach was tested on several datasets explained below in several examples.

### EXAMPLE #I

Human EpiDermFT 3D skin tissues were exposed to UVC to induce inflammation and then, in 24 h after exposure, treated with extracts of new cannabis lines via their addition to the tissue growth media and incubated for another 24 h. Untreated sample (“U”) had DMSO added to the media instead of extracts. Control (“C”) sample had not been exposed to UVC. All samples were collected 24 h after extracts were added and were used for mRNA extraction.

The high-throughput gene expression profiles were obtained using the Illumina NextSeq 500 mRNA next-generation sequencing platform NextSeq500. The mRNA fragment reads were aligned using HISAT v.2.0.5 and raw read counts were obtained with FeatureCounts v.1.6.1. Read counts were loaded into R and normalization was conducted using DESeq2 Bioconductor package (Love et al., 2014).

TABLE 1
CDEI testing results - EpiDermFT

| | | Number of | | p- | |
|---|---|---|---|---|---|
| Dataset | Type | profiles | t-value | value | CDEI |
| DMSO | Control (C) | 3 | 0 | 1 | — |
| UV | Untreated | 3 | 1.04 | 0.23 | 0.00 |
| | (U) | | | | |
| Extract #4 | Treated (T) | 5 | 0.67 | 0.50 | 0.22 |
| Extract #12 | Treated (T) | 5 | 2.06 | 0.04 | −0.33 |
| Extract #8 | Treated (T) | 5 | −0.19 | 0.85 | 0.69 |
| Extract #13 | Treated (T) | 5 | −1.04 | 0.30 | 0.00 |

In a ranking CDEI scores step 414, different drugs can be compared in their efficiency in a certain individual. Thus, in Table 1, the ranking order of the four extracts would be:

1) Extract #8, 2) Extract #4, 3) Extract #13, and 4) Extract #12. Of course, it can be seen that extract #12 would be detrimental and extract #13 would have no effect on this individual. In contrast, extract #4 would provide some effect and extract #8 would be best of all the four extracts for treating this specific individual.

### EXAMPLE #II

In this example, inflammation of tissues representing oral epithelium was established and the effect of extracts on the reversal of inflammation processes was evaluated. Human MatTek's 3D EpiOral tissues were equilibrated for 24 h (overnight) then culture media was replaced and incubated for another 24 h. Tissues were then exposed for 24 h to TNFα (40 ng/ml) to promote inflammation or to DMSO only. Tissues were then treated with various cannabis extracts that were added to the media for 24 h. Control sample was exposed to DMSO only and DMSO was added instead of extracts. Samples were then harvested for mRNA extraction. Sequencing and data analysis was performed as in Example #1.

TABLE 2
CDEI testing results - EpiOral

| Data set: Treated | Untreated Case | |
|---|---|---|
| Case vs Control | vs Control | CDEI |
| EpiO_#1_normal_vs_extract | EpiOral_normal_vs_disease | ** CDEI = 0.525 |
| | | Untreated case statistics = t(−2.78), p(0.006) |
| | | Treated case statistics = t(0.86), p(0.389) |
| EpiO_#2_normal_vs_extract | EpiOral_normal_vs_disease | ** CDEI = 0.869 |
| | | Untreated case statistics = t(−2.78), p(0.006) |
| | | Treated case statistics = t(−0.19), p(0.846) |
| EpiO_#3_normal_vs_extract | EpiOral_normal_vs_disease | ** CDEI = 0.979 |
| | | Untreated case statistics = t(−2.78), p(0.006) |
| | | Treated case statistics = t(−0.03), p(0.977) |
| EpiO_#4_normal_vs_extract | EpiOral_normal_vs_disease | ** CDEI = 0.157 |
| | | Untreated case statistics = t(−2.78), p(0.006) |
| | | Treated case statistics = t(−2.02), p(0.044) |
| EpiO_#5_normal_vs_extract | EpiOral_normal_vs_disease | ** CDEI = 0.899 |
| | | Untreated case statistics = t(−2.78), p(0.006) |
| | | Treated case statistics = t(−0.146), p(0.883) |
| EpiO_#6_normal_vs_extract | EpiOral_normal_vs_disease | ** CDEI = 0.628 |
| | | Untreated case statistics = t(−2.78), p(0.006) |
| | | Treated case statistics = t(−0.63), p(0.527) |
| EpiO_#7_normal_vs_extract | EpiOral_normal_vs_disease | ** CDEI = 0.808 |
| | | Untreated case statistics = t(−2.78), p(0.006) |
| | | Treated case statistics = t(−0.29), p(0.769) |
| EpiO_#8_normal_vs_extract | EpiOral_normal_vs_disease | ** CDEI = 0.101 |
| | | Untreated case statistics = t(−2.78), p(0.006) |
| | | Treated case statistics = t(−2.27), p(0.024) |
| EpiO_#9_normal_vs_extract | EpiOral_normal_vs_disease | ** CDEI = 0.883 |
| | | Untreated case statistics = t(−2.78), p(0.006) |
| | | Treated case statistics = t(−0.17), p(0.863) |

In a ranking CDEI scores step 414, different drugs can be compared in their efficiency in a certain individual. Thus, in Table 2, the ranking order of the extracts would be:

1) Extract #3, 2) Extract #5, 3) Extract #9, 4) Extract #2, 5) Extract #7, 6) Extract #6, 7) Extract #1, 8) Extract #4 and 9) Extract #8. As it can be seen, from Table 2, extracts #8 and #4 would have almost no effect, extracts #1 and #6 would have moderate effect, and extract #3 would have the strongest effect on recovery from inflammation. In fact, extract #3 demonstrates nearly perfect recovery from inflammation based on the restoration of gene expression to normal status.

### EXAMPLE #III

Human MatTek's 3D EpiIntestinal tissues were equilibrated for 24 h (overnight) then culture media was replaced and incubated for another 24 h. Tissues were then exposed for 24 h to TNFα (40 ng/ml) or to DMSO only. Tissues were then treated with various cannabis extracts that were added to the media for 24 h. Control sample was exposed to DMSO only and DMSO was added instead of extracts. Samples were then harvested for mRNA extraction. Sequencing and data analysis was performed as in Example #1.

TABLE 3
CDEI testing results_EpiIntestinal

| | Untreated Case vs | |
|---|---|---|
| Treated Case vs Control | Control | CDEI |
| Epiintes#1_normal_vs_extract | EpiIntestinal_normal_vs_disease | ** CDEI = 0.278 |
| | | Untreated case statistics = t(2.43), p(0.016) |
| | | Treated case statistics = t(−1.37), p(0.171) |
| Epiintes#2_normal_vs_extract | EpiIntestinal_normal_vs_disease | ** CDEI = 0.358 |
| | | Untreated case statistics = t(2.43), p(0.016) |
| | | Treated case statistics = t(1.15), p(0.251) |
| Epiintes#3_normal_vs_extract | EpiIntestinal_normal_vs_disease | ** CDEI = 0.259 |
| | | Untreated case statistics = t(2.43), p(0.016) |
| | | Treated case statistics = t(1.43), p(0.153) |
| Epiintes#4_normal_vs_extract | EpiIntestinal_normal_vs_disease | ** CDEI = 0.411 |
| | | Untreated case statistics = t(2.43), p(0.016) |
| | | Treated case statistics = t(1.02), p(0.311) |
| Epiintes#5_normal_vs_extract | EpiIntestinal_normal_vs_disease | ** CDEI = 0.844 |
| | | Untreated case statistics = t(2.43), p(0.016) |
| | | Treated case statistics = t(0.21), p(0.837) |
| Epiintes#6_normal_vs_extract | EpiIntestinal_normal_vs_disease | ** CDEI = 0.628 |
| | | Untreated case statistics = t(2.43), p(0.016) |
| | | Treated case statistics = t(−0.56), p(0.579) |
| Epiintes#10_normal_vs_extract | EpiIntestinal_normal_vs_disease | ** CDEI = 0.164 |
| | | Untreated case statistics = t(2.43), p(0.016) |
| | | Treated case statistics = t(1.75), p(0.082) |
| Epiintes#9_normal_vs_extract | EpiIntestinal_normal_vs_disease | ** CDEI = 0.219 |
| | | Untreated case statistics = t(2.43), p(0.016) |
| | | Treated case statistics = t(1.56), p(0.121) |
| Epiintes#11_normal_vs_extract | EpiIntestinal_normal_vs_disease | ** CDEI = 0.420 |
| | | Untreated case statistics = t(2.43), p(0.016) |
| | | Treated case statistics = t(0.99), p(0.322) |

In a ranking CDEI scores step 414, different drugs can be compared in their efficiency in a certain individual. Thus, in Table 3, the ranking order of the extracts would be:

1) Extract #5, 2) Extract #6, 3) Extract #11, 4) Extract #4, 5) Extract #2, 6) Extract #1, 7) Extract #3, 8) Extract #9 and 9) Extract #10. As it can be seen from Table 3, extracts #1, #10 and #9 would have very little effect, extracts #6, #3, #4 and #11 would have moderate effect, and extract #5 would have the strongest effect on recovery from inflammation. It can also be seen that when the effect of extracts on inflammation in oral and intestinal epithelial tissues is compared, certain extracts are similarly effective, like extract #5, certain extracts are effective only for one type of tissues, like extract #3 for oral epithelial tissues, and certain extracts, like extract #8 or #4, are not effective for any tissue tested.

In a repeat step 414, ranking CDEI scores is performed for all individuals for one or more candidate drugs. Preferably, this is performed for a number of drugs. In some cases, these drugs are extracts from different Cannabis strains.

FIG. 5 is a simplified bar chart of oral and intestinal CDEI scores for nine different experimental runs, in accordance with an embodiment of the present invention.

In some cases, the drugs are from hemp extracts. In some cases, these drugs are different extracts from the same Cannabis strains. In some cases, these drugs are derived drugs from different Cannabis strains.

In some cases, these drugs are combination therapies from different Cannabis strains. In some cases, these drugs are combinations of extracts from different Cannabis strains.

In some cases, these drugs are combinations of extracts from different Cannabis strains and at least one known pharmaceutical drug. In some cases, these drugs are combinations of extracts from different Cannabis strains and at least one known FDA-approved pharmaceutical drug. In some cases, the extracts are water-soluble. In other cases, the extracts are in one or more organic solvents. In yet other cases, the extracts are in one or more oils.

In some cases, these drugs are different extracts from the same Hemp strains. In some cases, these drugs are derived drugs from different Hemp strains. In some cases, these drugs are combination therapies from different Hemp strains. In some cases, these drugs are combinations of extracts from different Hemp strains. In some cases, these drugs are combinations of extracts from different Hemp strains and at least one known pharmaceutical drug. In some cases, these drugs are combinations of extracts from different Hemp strains and at least one known FDA-approved pharmaceutical drug. In some cases, the extracts are water-soluble. In other cases, the extracts are in one or more organic solvents. In yet other cases, the extracts are in one or more oils.

The compositions of the present invention may be provided in any suitable dosage form. These dosage forms may be injectable, infusible, inhalable, edible, oral or combinations thereof, as are known in the art. According to some embodiments, the dosage form is an oral dosage form. Oral dosage forms comprise liquids (solutions, suspensions, and emulsions), semi-solids (pastes), and solids (tablets, capsules, powders, granules, premixes, and medicated blocks).

In another embodiment, additional methods of administering the compositions of the invention comprise injectable dosage forms. In another embodiment, the injectable is administered intraperitoneally. In another embodiment, the injectable is administered intramuscularly. In another embodiment, the injectable is administered intradermally. In another embodiment, the injectable is administered intravenously. Each possibility represents a separate embodiment of the present invention.

In another embodiment, the compositions are administered by intravenous, intra-arterial, or intra-muscular injection of a liquid preparation. Suitable liquid formulations include solutions, suspensions, dispersions, emulsions, oils and the like. In another embodiment, the compositions are administered intravenously and are thus formulated in a form suitable for intravenous administration. In another embodiment, the pharmaceutical compositions are administered intra-arterially and are thus formulated in a form suitable for intra-arterial administration. In another embodiment, the compositions are administered intra-muscularly and are thus formulated in a form suitable for intra-muscular administration.

According to some additional embodiments of the present invention, a use of a pharmaceutical composition is provided for the preparation of a medicament suitable for administration to a human in a pharmaceutically effective amount, wherein the medicament is suitable for treating a disease or disorder in the human.

According to some additional embodiments of the present invention, the composition is suitable for oral, parenteral, transdermal, intra-venous or intra-muscular administration.

According to some embodiments, the composition is a slow-release composition. In some cases, the slow release composition is formulated for provision by at least one of an intravenous drip, a trans-dermal device and a slow-release oral formulation

The compositions of the present invention may comprise an additional active agent. The additional active agent is selected from the group consisting of active herbal extracts, acaricides, age spot and keratose removing agents, allergen, analgesics, local anesthetics, antiacne agents, antiallergic agents, antiaging agents, antibacterials, antibiotic agents, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

According to some embodiments the antibiotic agent is selected from the group consisting of beta-lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, antibiotic steroids, sulfonamides, tetracycline, dicarboxylic acids, antibiotic metals including antibiotic metal ions, oxidizing agents, substances that release free radicals and/or active oxygen, cationic antimicrobial agents, quaternary ammonium compounds, biguanides, triguanides, bisbiguanides and analogs and polymers thereof, naturally occurring antibiotic compounds, including antibiotic plant oils and antibiotic plant extracts and any one of the following antibiotic compounds: chlorhexidine acetate, chlorhexidine gluconate and chlorhexidine hydrochloride, picloxydine, alexidine, polihexanide, chlorproguanil hydrochloride, proguanil hydrochloride, metformin hydrochloride, phenformin, buformin hydrochloride, abomycin, acetomycin, acetoxycycloheximide, acetylnanaomycin, an actinoplanes sp. compound, actinopyrone, aflastatin, albacarcin, albacarcin, albofungin, albofungin, alisamycin, alpha-R,S-methoxycarbonylbenzylmonate, altromycin, amicetin, amycin, amycin demanoyl compound, amycine, amycomycin, anandimycin, anisomycin, anthramycin, anti-syphilis imune substance, anti-tuberculosis imune substance, antibiotic from Escherichia coli, antibiotics from Streptomyces refuineus, anticapsin, antimycin, aplasmomycin, aranorosin, aranorosinol, arugomycin, ascofuranone, ascomycin, ascosin, Aspergillus flavus antibiotic, asukamycin, aurantinin, an Aureolic acid antibiotic substance, aurodox, avilamycin, azidamfenicol, azidimycin, bacillaene, a Bacillus larvae antibiotic, bactobolin, benanomycin, benzanthrin, benzylmonate, bicozamycin, bravomicin, brodimoprim, butalactin, calcimycin, calvatic acid, candiplanecin, carumonam, carzinophilin, celesticetin, cepacin, cerulenin, cervinomycin, chartreusin, chloramphenicol, chloramphenicol palmitate, chloramphenicol succinate sodium, chlorflavonin, chlorobiocin, chlorocarcin, chromomycin, ciclopirox, ciclopirox olamine, citreamicin, cladosporin, clazamycin, clecarmycin, clindamycin, coliformin, collinomycin, copiamycin, corallopyronin, corynecandin, coumermycin, culpin, cuprimyxin, cyclamidomycin, cycloheximide, dactylomycin, danomycin, danubomycin, delaminomycin, demethoxyrapamycin, demethylscytophycin, dermadin, desdamethine, dexylosyl-benanomycin, pseudoaglycone, dihydromocimycin, dihydronancimycin, diumycin, dnacin, dorrigocin, dynemycin, dynemycin triacetate, ecteinascidin, efrotomycin, endomycin, ensanchomycin, equisetin, ericamycin, esperamicin, ethylmonate, everninomicin, feldamycin, flambamycin, flavensomycin, florfenicol, fluvomycin, fosfomycin, fosfonochlorin, fredericamycin, frenolicin, fumagillin, fumifungin, funginon, fusacandin, fusafungin, gelbecidine, glidobactin, grahamimycin, granaticin, griseofulvin, griseoviridin, grisonomycin, hayumicin, hayumicin, hazymicin, hedamycin, heneicomycin, heptelicid acid, holomycin, humidin, isohematinic acid, karnatakin, kazusamycin, kristenin, L-dihydrophenylalanine, a L-isoleucyl-L-2-amino-4-(4′-amino-2′,5′-cyclohexadienyl) derivative, lanomycin, leinamycin, leptomycin, libanomycin, lincomycin, lomofungin, lysolipin, magnesidin, manumycin, melanomycin, methoxycarbonylmethylmonate, methoxycarbonylethylmonate, methoxycarbonylphenylmonate, methyl pseudomonate, methylmonate, microcin, mitomalcin, mocimycin, moenomycin, monoacetyl cladosporin, monomethyl cladosporin, mupirocin, mupirocin calcium, mycobacidin, myriocin, myxopyronin, pseudoaglycone, nanaomycin, nancimycin, nargenicin, neocarcinostatin, neoenactin, neothramycin, nifurtoinol, nocardicin, nogalamycin, novobiocin, octylmonate, olivomycin, orthosomycin, oudemansin, oxirapentyn, oxoglaucine methiodide, pactacin, pactamycin, papulacandin, paulomycin, phaeoramularia fungicide, phenelfamycin, phenyl, cerulenin, phenylmonate, pholipomycin, pirlimycin, pleuromutilin, a polylactone derivative, polynitroxin, polyoxin, porfiromycin, pradimicin, prenomycin, Prop-2-enylmonate, protomycin, Pseudomonas antibiotic, pseudomonic acid, purpuromycin, pyrinodemin, pyrrolnitrin, pyrrolomycin, amino, chloro pentenedioic acid, rapamycin, rebeccamycin, resistomycin, reuterin, reveromycin, rhizocticin, roridin, rubiflavin, naphthyridinomycin, saframycin, saphenamycin, sarkomycin, sarkomycin, sclopularin, selenomycin, siccanin, spartanamicin, spectinomycin, spongistatin, stravidin, streptolydigin, streptomyces arenae antibiotic complex, streptonigrin, streptothricins, streptovitacin, streptozotocine, a strobilurin derivative, stubomycin, sulfamethoxazol-trimethoprim, sakamycin, tejeramycin, terpentecin, tetrocarcin, thermorubin, thermozymocidin, thiamphenicol, thioaurin, thiolutin, thiomarinol, thiomarinol, tirandamycin, tolytoxin, trichodermin, trienomycin, trimethoprim, trioxacarcin, tyrissamycin, umbrinomycin, unphenelfamycin, urauchimycin, usnic acid, uredolysin, variotin, vermisporin, verrucarin, metronidazole, erythromycin and analogs, salts and derivatives thereof.

According to some embodiments, the additional active agent is selected from the group consisting of alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, diflurosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, .alpha.-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters and salts thereof.

According to some embodiments, the compositions of the present invention further comprise an antiviral agent Suitable antiviral agents include but are not limited to, acyclovir, gancyclovir, ribavirin, amantadine, rimantadine nucleoside-analog reverse transcriptase inhibitors, such as zidovudine, didanosine, zalcitabine, tavudine, lamivudine and vidarabine, non-nucleoside reverse transcriptase inhibitors, such as nevirapine and delavirdine, protease inhibitors, such as saquinavir, ritonavir, indinavir and nelfinavir, and interferons and derivatives, esters, salts and mixtures thereof.

According to some embodiments, the compositions of the present invention further comprise a chemotherapeutic agent. Suitable chemotherapeutic agents include but are not limited to daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.

According to some embodiments, the compositions of the present invention further comprise a corticosteroid. Suitable corticosteroids include but are not limited to alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, diflurosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, .alpha.-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters, salts and mixtures thereof.

According to some embodiments, the compositions of the present invention further comprise an analgesic. Suitable analgesics include but are not limited to benzocaine, butamben picrate, dibucaine, dimethisoquin, dyclonine, lidocaine, pramoxine, tetracaine, salicylates and derivatives, esters, salts and mixtures thereof.

According to some embodiments, the compositions of the present invention further comprise a non-steroidal anti-inflammatory agent. Suitable non-steroidal anti-inflammatory agent include but are not limited to azelaic acid, oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof.

In a ranking different Cannabis strains step 418, the outcomes of the step 414 and 416 are compared to provide a ranking of efficiency of the different strains' extracts for different individuals and for different diseases or disorders.

Using the methodology of the present invention, one or more drugs can be defined that provide the best predicted outcomes for a certain patient, based on his/her genotypic/phenotypic profile.

Moreover, using the methodology of the present invention, one or more drugs can be defined that provide the best predicted outcomes for a group of patients suffering from the same disease.

Additionally, the methods of the present invention may allow the use of one or more drugs, which provide the best predicted outcomes for a group of patients of the same ethnicity, suffering from the same disease.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

### REFERENCES

Borisov, N., Suntsova, M., Sorokin, M., Garazha, A., Kovalchuk, O., Aliper, A., Ilnitskaya, E., Lezhnina, K., Korzinkin, M., Tkachev, V., et al. (2017). Data aggregation at the level of molecular pathways improves stability of experimental transcriptomic and proteomic data. Cell Cycle Georget. Tex 16, 1810-1823.
Buzdin, A. A., Zhavoronkov, A. A., Korzinkin, M. B., Venkova, L. S., Zenin, A. A., Smirnov, P. Y., and Borisov, N. M. (2014). Oncofinder, a new method for the analysis of intracellular signaling pathway activation using transcriptomic data. Front. Genet. 5.
Draghici, S., Khatri, P., Tarca, A. L., Amin, K., Done, A., Voichita, C., Georgescu, C., and Romero, R. (2007). A systems biology approach for pathway level analysis. Genome Res. 17, 1537-1545.
Even, S., and Guy Even (2012). Graph algorithms (Cambridge, N.Y.: Cambridge University Press).
Gao, S., and Wang, X. (2007). TAPPA: topological analysis of pathway phenotype association. Bioinforma. Oxf. Engl. 23, 3100-3102.
Ibrahim, M. A.-H., Jassim, S., Cawthorne, M. A., and Langlands, K. (2012). A topology-based score for pathway enrichment. J. Comput. Biol. J. Comput. Mol. Cell Biol. 19, 563-573.
Love, M. I., Huber, W., and Anders, S. (2014). Moderated estimation of fold change and dispersion for RNA-seq data with DESeq2. Genome Biol. 15, 550.
Tarca, A. L., Draghici, S., Khatri, P., Hassan, S. S., Mittal, P., Kim, J.-S., Kim, C. J., Kusanovic, J. P., and Romero, R. (2009). A novel signaling pathway impact analysis. Bioinforma. Oxf. Engl. 25, 75-82.

1. A method for ranking efficiency of Cannabis drugs, the method comprising:
a. calculating a Signaling Pathway Impact analysis (SPIA) for each drug for each biological pathway;
b. determining a mean weighted SPIA_{μ} wherein SPIA_{μ}=mean(SPIA)·wₚ;
c. calculating a cannabis drug efficiency index (CDEI) for each drug for a specific disease, wherein CDEI=2 ((|t_{U}|/(|t_{T}|+|t_{U}|)−0.5); and
d. ranking said drugs according to highest CDEI for a group of individual patients.

2. A method according to claim 1, further comprising treating an individual patient suffering from said specific disease with at least one drugs of one or more high CDEIs, wherein said high CDEI>0.

3. A method according to claim 2, wherein said at least one drug alleviates, cures or attenuates said specific disease.

4. A method according to claim 3, wherein said high CDEI>0 is a highest ranking CDEI.

5. A method according to claim 3, wherein said disease is proliferative disease or disorder.

6. A method according to claim 4, wherein said proliferative disease or disorder is cancer.

7. A method according to claim 1, wherein said CDEI>=0.2.

8. A method according to claim 1, wherein said CDEI>=0.5 and <1.

9. A method according to claim 1, wherein said wₚ=([number of case samples with positive SPIA score]/[total number of case samples).

10. A method according to claim 1, wherein said wₚ=([number of case samples with negative SPIA score]/[total number of case samples).

11. A bioinformatics method for ranking Cannabis drugs, the method comprising:
a. calculating a Signaling Pathway Impact analysis (SPIA) for each drug for each biological pathway;
b. determining a mean weighted SPIA_{μ} wherein SPIA_{μ}=mean(SPIA)·wₚ;
c. calculating a cannabis drug efficiency index (CDEI) for each drug for a specific disease, wherein CDEI=2 ((|t_{U}|/(|t_{T}|+|t_{U}|)−0.5); and
d. ranking said drugs according to highest CDEI for a group of individual patients.

12. A method according to claim 11, wherein said method is performed on a plurality of ethnic groups to determine an optimized ranking of said disease-protective drugs for each ethnic group.

13. A method according to claim 11, wherein said method is performed for an individual to determine an optimized ranking of said disease-protective drugs for said individual.

14. A method according to claim 11, wherein said disease is cancer.

15. A method according to claim 11, wherein said biological pathways are signaling pathways.

16. A method according to claim 11, wherein data is obtained from studies on samples of said individual patients.

17. A method according to claim 16, wherein said samples are bodily samples selected from the group consisting of a blood sample, a urine sample, a biopsy, a hair sample, a nail sample, a breathe sample, a saliva sample and a skin sample.
a. patient suffering for a disease.

18. A method for treating an individual suffering from a disease or disorder, the method comprising;
a. choosing a drug with the highest cannabis drug efficiency index (CDEI) according to the method of claim 11; and
b. administering said drug to said individual in a pharmaceutically effective amount.

19. A method according to claim 18, further comprising:
c. administering a second drug to said individual, wherein said second drug is suitable for treating said disease or said disorder.

20. A method according to claim 19, wherein said disease is a proliferative disease and wherein said second drug is a chemotherapeutic agent selected from the group consisting of: daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.
